(19) United States
(12) Patent Application Publication
(10) Pub. No.: US 2012/0157873 A1
(43) Pub. Date: Jun. 21, 2012

(54) WAKE-UP DEVICE FOR NAPPING TIME

(76) Inventors: Shen-Fu LIANG, Tainan County (TW); Yu-Han Hu, Kaohsiung County (TW); Ying-Huang Chen, Kaohsiung County (TW)

(21) Appl. No.: 13/327,081

(22) Filed: Dec. 15, 2011

(30) Foreign Application Priority Data
Dec. 17, 2010 (TW) 099144589

(51) Int. Cl. A61B 5/0476

(52) U.S. Cl. 600/544

(57) ABSTRACT

A wake-up device, used for awaking a user in a nap or a short-term sleep, includes an electroencephalogram (EEG) sensor, a processing module and a wake-up module. The EEG sensor is disposed on the user's head for sensing an EEG signal from the user. The processing module is coupled with the EEG sensor, and receives and processes the EEG signal. If the user is determined in the sleep stage 2 according to the EEG signal, the processing module outputs a wake-up signal. The wake-up module is coupled to the processing module for receiving the wake-up signal, and awakes the user according to the wake-up signal.

### CROSS REFERENCE TO RELATED APPLICATIONS

This Non-provisional application claims priority under 35 U.S.C. §119(a) on patent application Ser. No(s). 099144589 filed in Taiwan, Republic of China on Dec. 17, 2010, the entire contents of which are hereby incorporated by reference.

### BACKGROUND OF THE INVENTION

1. Field of Invention

The present invention relates to a wake-up device and, in particular, to a wake-up device applied to a user in a nap or a short-term sleep.

2. Related Art

Sleeping is a very important behavior of humans. A good sleeping can help people to restore their energy, release their pressure, improve their memory ability, and thus make them healthier.

However, according to the change of life style of human beings, the quality and quantity of sleeping become worse than ever. If a person can not have enough sleep, he/she may show some bad responses. For example, this person may have good activity in the morning, but become sleepy in the afternoon. Besides, he/she may have unstable emotion, lose the ability of contingency, and lower the working performance.

It is known that a nap can help to recovery. After the hard work in the morning and the delicious lunch, people may feel a little tired. At this time, if a nap is available, people can become more active in the afternoon. However, a longer nap may cause a headache or dizzy and may affect the sleeping that night. On the contrary, a short nap can not achieve the desired recovery effect.

Therefore, it is an important subject of the present invention to provide a wake-up device that can awake the user at a proper timing so as to achieve the high performance nap or short-term sleep.

### SUMMARY OF THE INVENTION

In view of the foregoing subject, an object of the present invention is to provide a wake-up device applied to a user in nap or a short-term sleep, which can awake the user at a specific time point after sleeping so as to achieve the high performance nap or short-term sleep.

Many researches support that a person can have the best situation after a nap or short-term sleep if he/she is awaked in the sleep stage 2. Accordingly, the present invention is designed to awake the user after the user enters the sleep stage 2.

To achieve the above object, the present invention discloses a wake-up device used for awaking a user in a nap or a short-term sleep. The wake-up device includes an electroencephalogram (EEG) sensor, a processing module and a wake-up module. The EEG sensor is disposed on the user's head for sensing an EEG signal from the user. The processing module is coupled with the EEG sensor, and receives and processes the EEG signal. If the user is determined in the sleep stage 2 according to the EEG signal, the processing module outputs a wake-up signal. The wake-up module is coupled to the processing module for receiving the wake-up signal, and awakes the user according to the wake-up signal.

In one embodiment of the present invention, the processing module further includes a filter-amplifying unit, an analog-to-digital conversion unit, an operation unit, and a timing unit. The filter-amplifying unit is coupled with the EEG sensor for filtering and amplifying the received EEG signal. The analog-to-digital conversion unit is coupled with the EEG sensor for performing an analog-to-digital conversion with the received EEG signal. The operation unit is coupled with the EEG sensor for analyzing the received EEG signal to judge whether the user is in the sleep stage 2 or not. The timing unit is coupled with the operation unit for counting a time period after the user enters the sleep stage 2.

In one embodiment of the present invention, the operation unit retrieves a frequency, a cycle, an amplitude, or a waveform of the EEG signal, and compares the retrieved frequency, cycle, amplitude, or waveform with a frequency, a cycle, an amplitude, or a waveform of a specific signal. Herein, the specific signal is a sleep spindle wave or a slow wave (a wave).

In one embodiment of the present invention, when the time period counted by the timing unit excesses a specific time period (e.g. 5 to 15 minutes), the operation unit outputs the wake-up signal.

To achieve the above object, the present invention also discloses a wake-up method applied to a user in a nap or a short-term sleep. The wake-up method includes the following steps of: sensing an electroencephalogram (EEG) signal from the user; judging whether the user is in a sleep stage 2 according to the EEG signal; and if the user is judged in the sleep stage 2, outputting a wake-up signal for awaking the user.

In one embodiment of the present invention, the wake-up method further includes the following steps of: retrieving a frequency, a cycle, an amplitude, or a waveform of the EEG signal; and comparing the retrieved frequency, cycle, amplitude, or waveform with a frequency, a cycle, an amplitude, or a waveform of a specific signal for judging whether the user is in the sleep stage 2. Herein, the specific signal is a sleep spindle wave or a slow wave.

In one embodiment of the present invention, the wake-up method further includes the following steps of: counting a time period after the user enters the sleep stage 2; and outputting the wake-up signal when the counted time period excesses a specific time period.

As mentioned above, the wake-up device of the present invention can sense an EEG signal from the user in a nap or a short-term sleep by an EEG sensor, and then analyze the EEG signal to judge whether the user is in a sleep stage 2 or not. If determining that the user is in the sleep stage 2, a wake-up signal is outputted for awaking the user. This feature allows the user to have a high performance nap or short-term sleep.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a block diagram of a wake-up device according to a preferred embodiment of the present invention;

FIG. 2 is another block diagram of the wake-up device according to the preferred embodiment of the present invention;

FIG. 3 is a flow chart of a wake-up method applied to a user in a nap or a short-term sleep according to the preferred embodiment of the present invention; and

FIG. 4 is a flow chart showing the details of the step of judging whether the user is in a sleep stage 2.

### DETAILED DESCRIPTION OF THE INVENTION

The present invention will be apparent from the following detailed description, which proceeds with reference to the accompanying drawings, wherein the same references relate to the same elements.

FIG. 1 is a block diagram of a wake-up device 1 according to a preferred embodiment of the present invention. Referring to FIG. 1, the wake-up device 1 used for awaking a user 11 includes an electroencephalogram (EEG) sensor 12, a processing module 13 and a wake-up module 14. The EEG sensor 12 is disposed on the head of the user 11 for sensing an EEG signal from the user 11. The processing module 13 is coupled with the EEG sensor 12 for receiving and processing the EEG signal of the user 11. If the user 11 is determined in the sleep stage 2 after a specific time period, the processing module 13 outputs a wake-up signal. The wake-up module 14 is coupled to the processing module 13 for receiving the wake-up signal, and then outputs a sound, a vibration, a flash of other signals for awaking the user 11 according to the wake-up signal. In this embodiment, the wake-up module 14 outputs a sound to awake the user 11, but it is also possible to use any available method to awake the user 11.

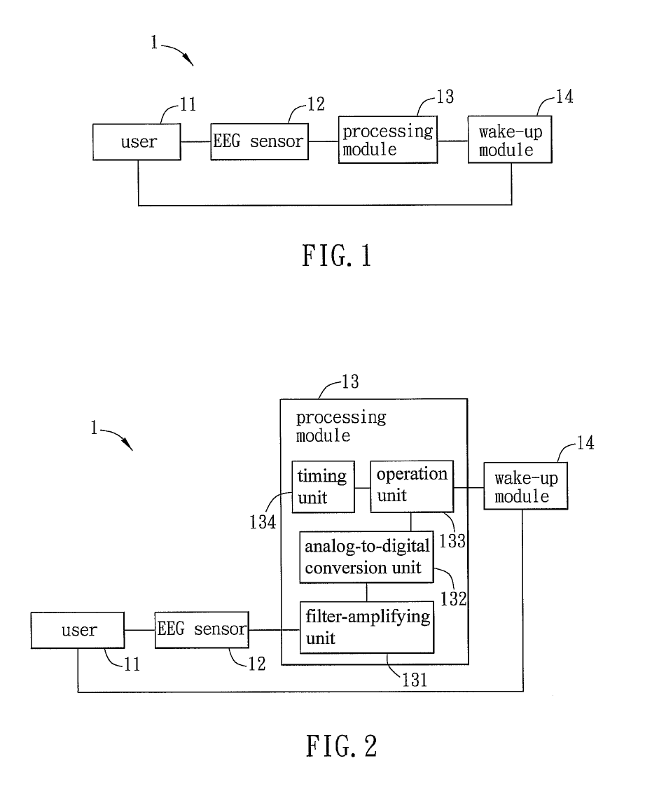

FIG. 2 is another block diagram of the wake-up device 1 according to the preferred embodiment of the present invention. Referring to FIG. 2, the processing module 13 further includes a filter-amplifying unit 131, an analog-to-digital conversion unit 132, an operation unit 133, and a timing unit 134. The filter-amplifying unit 131 is coupled with the EEG sensor 12 for filtering and amplifying the received EEG signal. The analog-to-digital conversion unit 132 is coupled with the EEG sensor 12 for performing an analog-to-digital conversion with the filtered and amplified EEG signal. In this embodiment, the analog-to-digital conversion unit 132 is indirectly coupled with the EEG sensor 12 and directly coupled with the filter-amplifying unit 131. The operation unit 133 is indirectly coupled with the EEG sensor 12 and directly coupled with the analog-to-digital conversion unit 132. The operation unit 133 is configured to receive the converted EGG signal and analyze it to judge whether the user 11 is in the sleep stage 2 or not. If the user 11 is in the sleep stage 2, the operation unit 133 outputs a wake-up signal. Besides, the timing unit 134 is coupled with the operation unit 133 for counting a time period after the operation unit 133 determines the user 11 has entered the sleep stage 2. After a specific time period since the user 11 entered the sleep stage 2, the operation unit 133 starts to output the wake-up signal.

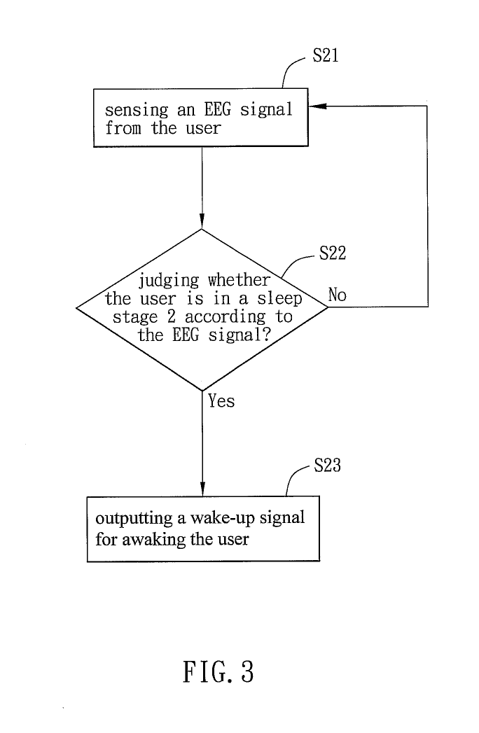

FIG. 3 is a flow chart of a wake-up method applied to a user in a nap or a short-term sleep according to the preferred embodiment of the present invention. The wake-up method can be carried out by the above-mentioned wake-up device 1. The wake-up method includes the following steps S21 to S23. The step S21 is to sense an electroencephalogram (EEG) signal from the user 11. The step S22 judges whether the user 11 is in a sleep stage 2 according to the EEG signal. If the step S22 determines that the user 11 is not in a sleep stage 2, this method goes back to the step S21. If the step S22 determines that the user 11 is in the sleep stage 2, the step S23 is performed to output a wake-up signal for awaking the user 11.

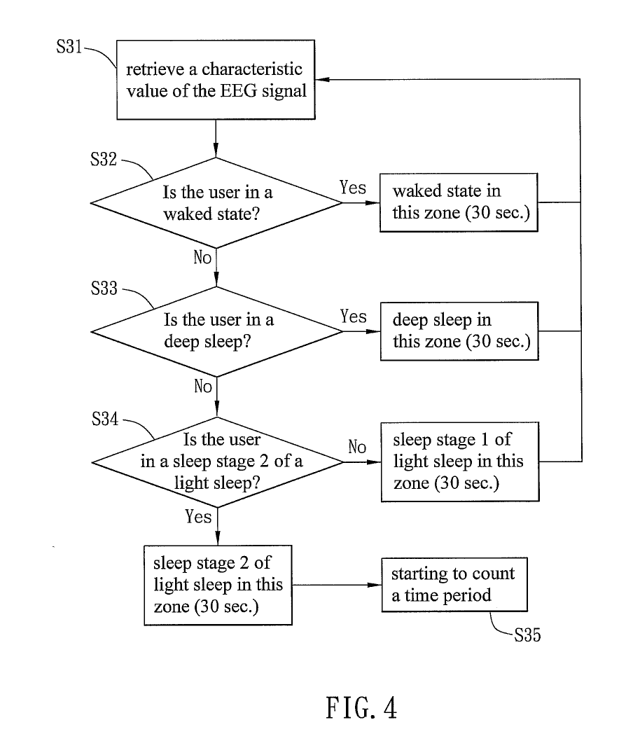

In addition, the step S22 includes the following detailed steps S31 to S35 as shown in FIG. 4. The step S31 is to retrieve a frequency, a cycle, an amplitude, or a waveform of the EEG signal. For example, the EEG signal is divided into several zones (each zone is 30 seconds), and the characteristic value (e.g. frequency, cycle, amplitude, or waveform) of each zone is retrieved. Next, the step S32 determines whether the user 11 is in a waked state. If yes, the procedure goes back to the step S31; otherwise, if not, the procedure goes to the step S33. In the step S32, it is possible to determine whether the user 11 is in the waked state by comparing the retrieved characteristic value with a corresponding characteristic value of a specific signal (e.g. a wave). Then, the step S33 determines whether the user is in a deep sleep or not. If yes, the procedure goes back to the step S31; otherwise, if not, the procedure goes to the step S34. In the step S33, the retrieved characteristic value is compared with the corresponding characteristic value of a specific signal (e.g. slow wave) so as to determine whether the user is in a deep sleep or not. The step S34 is to determine whether the user is in a sleep stage 2 of a light sleep or not. If not, which means the user is in a sleep stage 1 of a light sleep, the procedure goes back to the step S31. Otherwise, if yes, the procedure goes back to the step S35 for starting to count a time period. In the step S34, the retrieved characteristic value is compared with the corresponding characteristic value of a specific signal (e.g. sleep spindle wave) so as to determine whether the user is in a sleep stage 2 or not.

In summary, the wake-up device of the present invention can sense an EEG signal from the user in a nap or a short-term sleep by an EEG sensor, and then analyze the EEG signal to judge whether the user is in a sleep stage 2 or not. If determining that the user is in the sleep stage 2, a wake-up signal is outputted for awaking the user. This feature allows the user to have a high performance nap or short-term sleep.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A wake-up device, used for awaking a user in a nap or a short-term sleep, the wake-up device comprising:
an electroencephalogram (EEG) sensor disposed on the user's head for sensing an EEG signal from the user;
a processing module coupled with the EEG sensor for receiving the EEG signal and then processes the EEG signal, wherein if the user is determined in a sleep stage 2 according to the EEG signal, the processing module outputs a wake-up signal; and
a wake-up module coupled to the processing module for receiving the wake-up signal, and awaking the user according to the wake-up signal.

2. The wake-up device of claim 1, wherein the processing module comprises:
a filter-amplifying unit coupled with the EEG sensor for filtering and amplifying the received EEG signal.

3. The wake-up device of claim 1, wherein the processing module comprises:
an analog-to-digital conversion unit coupled with the EEG sensor for performing an analog-to-digital conversion with the received EEG signal.

4. The wake-up device of claim 1, wherein the processing module comprises:
an operation unit coupled with the EEG sensor for analyzing the received EEG signal to judge whether the user is in the sleep stage 2 or not.

5. The wake-up device of claim 4, wherein the operation unit retrieves a frequency, a cycle, an amplitude, or a waveform of the EEG signal, and compares the retrieved frequency, cycle, amplitude, or waveform with a frequency, a cycle, an amplitude, or a waveform of a specific signal.

6. The wake-up device of claim 5, wherein the specific signal is a sleep spindle wave or a slow wave.

7. The wake-up device of claim 4, wherein the processing module further comprises:
a timing unit coupled with the operation unit for counting a time period after the user enters the sleep stage 2.

8. The wake-up device of claim 7, wherein when the time period counted by the timing unit excesses a specific time period, the operation unit outputs the wake-up signal.

9. A wake-up method, applied to a user in a nap or a short-term sleep, the wake-up method comprising the steps of:
sensing an electroencephalogram (EEG) signal from the user;
judging whether the user is in a sleep stage 2 according to the EEG signal; and
if the user is judged in the sleep stage 2, outputting a wake-up signal for awaking the user.

10. The wake-up method of claim 9, further comprising steps of:
retrieving a frequency, a cycle, an amplitude, or a waveform of the EEG signal; and
comparing the retrieved frequency, cycle, amplitude, or waveform with a frequency, a cycle, an amplitude, or a waveform of a specific signal for judging whether the user is in the sleep stage 2.

11. The wake-up method of claim 10, wherein the specific signal is a sleep spindle wave or a slow wave.

12. The wake-up method of claim 9, further comprising steps of:
counting a time period after the user enters the sleep stage 2; and
outputting the wake-up signal when the counted time period excesses a specific time period.
